(19) United States
(12) Patent Application Publication
(10) Pub. No.: US 2004/0126364 A1
(43) Pub. Date: Jul. 1, 2004

(54) Treatment for bone disorders

(76) Inventors: Hugh S. Keeping, Bristol, RI (US); Jonathan S. Reichner, North Providence, RI (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/683,516

(22) Filed: Oct. 9, 2003

Related U.S. Application Data
(63) Continuation of application No. 09/507,239, filed on Feb. 18, 2000, now abandoned.

(51) Int. Cl. A61K 48/00; C12N 5/08; A61K 31/65

(52) U.S. Cl. 424/93.21; 514/44; 514/152; 435/366

(57) ABSTRACT

The invention provides compositions and methods to deliver an anti-inflammatory composition, e.g., recombinant human interleukin-4 (rhIL-4), to build (or rebuild) bone tissue. The composition is produced from living osteoprogenitor cells (OPCs) or odontoprogenitor cells.

### BACKGROUND OF THE INVENTION

The invention relates to bone loss disorders. Catastrophic bone loss, i.e., osteolysis, is a debilitating pathological consequence of a spectrum of disease states including rheumatoid arthritis, osseous metastasis, aseptic prosthetic loosening and periodontitis. Rheumatoid arthritis (RA) is a chronic inflammatory disease which often results in long term disability and increased mortality.

### SUMMARY OF THE INVENTION

The invention provides compositions and methods to deliver an anti-inflammatory composition, e.g., recombinant human interleukin-4 (rhIL-4), to build (or rebuild) bone tissue. The composition is produced from living osteoprogenitor cells (OPCs) or odontoprogenitor cells. The cells contain a genetically-engineered viral or non-viral plasmid vector containing a regulatable, inducible, osteoblast-specific promoter to direct expression of an anti-inflammatory polypeptide at specific sites of implantation in bone to inhibit osteolysis. For example, a bone stromal cell is isolated from autologous or allogeneic periodontal ligament and manipulated ex vivo prior to implantation into a recipient patient. Stromal cells are cultured in the presence of extracellular matrix (ECM) components to differentiate into odontoprogenitor cells. For example, ECM contains bone morphogenic proteins (BMPs) such as BMP-6. Induction of differentiation to progenitor cells is carried out before or after genetic manipulation of the cells.

Preferably, the nucleic acid with which the cells are transfected or transduced encodes an anti-inflammatory cytokine or anti-inflammatory fragment of the cytokine. For example, the cytokine is interleukin-4 (IL-4). The nucleic acid encodes a polypeptide containing the amino acid sequence of SEQ ID NO: 1; for example, the nucleic acid contains the coding region of the nucleotide sequence of SEQ ID NO:2.

TABLE 1
Human IL-4 Amino Acid Sequence

MGLTSQLLPPLFFLLACAGNFVHGHKCDITLQEIIKTLNSLTEQKTLCTE
LTVTDIFAASKNTTEKETFCRAATVLRQFYSHHEKDTRCLGATAQQFHRH
KQLIRFLKRLDRNLWGLAGLNSCPVKEANQSTLENFLERLKTIMREKYSK
CSS

(SEQ ID NO:2, GENBANK ™ Accession No. M13982)

TABLE 2
Human IL-4 Nucleotide Sequence

| | |
|---|---|
| 1 | gatcgttagc ttctcctgat aaactaattg cctcacattg tcactgcaaa tcgacaccta |
| 61 | ttaatgggtc tcacctccca actgcttccc cctctgttct tcctgctagc atgtgccggc |
| 121 | aactttgtcc acggacacaa gtgcgatatc accttacagg agatcatcaa aactttgaac |
| 181 | agcctcacag agcagaagac tctgtgcacc gagttgaccg taacagacat ctttgctgcc |
| 241 | tccaagaaca caactgagaa ggaaaccttc tgcagggctg cgactgtgct ccggcagttc |
| 301 | tacagccacc atgagaagga cactcgctgc ctgggtgcga ctgcacagca gttccacagg |
| 361 | cacaagcagc tgatccgatt cctgaaacgg ctcgacagga acctctgggg cctggcgggc |
| 421 | ttgaattcct gtcctgtgaa ggaagccaac cagagtacgt tggaaaactt cttggaaagg |
| 481 | ctaaagacga tcatgagaga gaaatattca aagtgttcga gctgaatatt ttaatttatg |
| 541 | agtttttgat agctttattt tttaagtatt tatatattta taactcatca taaaataaag |
| 601 | tatatataga atct |

SEQ ID NO:2, GENBANK ™ Accession No. M13982; coding sequences span nucleotides 64-525; signal peptide encoded by nucleotides 64-135).

Alternatively, the cells contain a nucleic acid encoding an IL-4 fragment, agonist or mutant. The fragment, agonist or mutant has anti-inflammatory activity. For example, the mutant contains a mutation in the region of IL-4 which is involved in binding to IL-2R gamma, e.g., Arg 21 is changed to a Glu residue. Sequences which differ from the coding sequence of SEQ ID NO:2 hybridize under stringent conditions, with all or part of the reference sequence and encode an anti-inflammatory polypeptide. Promoter or transcriptional regulatory elements which differ from a reference sequence hybridize under stringent conditions to a nucleic acid having the reference sequence and retain transcription regulatory function, e.g., cell specificity, of the reference sequence. For example, the nucleic acid may contain one or more sequence modifications in relation to a reference sequence. Such modifications may be obtained by mutation, deletion and/or addition of one or more nucleotides compared to the reference sequence. Modifications are introduced to alter the activity of the regulatory sequence, e.g., to improve promoter activity, to suppress a transcription inhibiting region, to make a promoter constitutive or regulatable or vice versa. Modifications are also made to introduce a restriction site to facilitate subsequent cloning steps, or to eliminate the sequences which are not essential to the transcriptional activity. Preferably, a modified sequence is at least 70% (more preferably at least 80%, more preferably at least 90%, more preferably at least 95%, more preferably at least 99%) identical to a reference sequence. The modifications do not substantially alter the biological function of a polypeptide or the cell-specificity of transcription promoter function associated with the reference sequence.

Nucleotide and amino acid comparisons are carried out using the Lasergene software package (DNASTAR, Inc., Madison, Wis.). The MegAlign module used was the Clustal V method (Higgins et al., 1989, CABIOS 5(2):151-153). The parameters used were gap penalty 10, gap length penalty 10.

Alternatively, nucleic acids which differ from a given reference sequence hybridize at high stringency to a strand of DNA having the reference sequence, or the complement thereof. Hybridization is carried out using standard techniques, such as those described in Ausubel et al. (Current Protocols in Molecular Biology, John Wiley & Sons, 1989). “High stringency” refers to nucleic acid hybridization and wash conditions characterized by high temperature and low salt concentration, i.e., hybridization at 42 degrees C., and in 50% formamide; a first wash at 65 degrees C., 2×SSC, and 1% SDS; followed by a second wash at 65 degrees C. and 0.2%×SSC, 0.190 SDS. Lower stringency conditions suitable for detecting DNA sequences having about 50% sequence identity to a reference gene or sequence are detected by hybridization at 42 degrees C. in the absence of formamide; a first wash at 42 degrees C. in 6×SSC and 1% SDS; and a second wash at 50 degrees C. in 6×SSC and 1% SDS.

A heterologous nucleic acid encoding a polypeptide (e.g., IL-4) is operably linked to an osteoblast-specific promoter such as an osteocalcin promoter sequence (e.g., a nucleic acid containing the nucleotide sequence of SEQ ID NO:3) or a bone sialoprotein promoter sequence (nucleotides 1-2472 of SEQ ID NO:4) or dentin sialoprotein promoter sequence (SEQ ID NO:6 and/or 7).

TABLE 3
Human Osteocalcin Regulatory Region

| | |
|---|---|
| 1 | ttctcctgtc cggatgcgca gggcagggct gaccgtcgag ctgcacccac agcaggctgc |
| 61 | ctttggtgac tcaccgggtg aacgggggca ttgcgaggca tcccctccct gggtttggct |
| 121 | cctgcccacg ggcctgacag tagaaatcac aggctgtgag acagctggag cccagctctg |
| 181 | cttgaaccta ttttaggtct ctgatccccg cttcctcttt agactcccct agagctcagc |
| 241 | cagtgctcaa cctgaggctg ggggtctctg aggaagagtg agttggagct gaggggtctg |
| 301 | gggctgtccc ctgagagagg ggccagaggc agtgtcaaga gccgggcagt ctgattgtgg |
| 361 | ctcaccctcc atcactccca ggggcccctg gcccagcagc cgcagctccc aaccacatat |
| 421 | cctctggggt ttggcctacg gagctggggc ggatgacccc caaatagccc tggcagattc |
| 481 | cccctagacc cgcccgcacc atggtcaggc atgcccctcc tcatcgctgg gcacagccca |
| 541 | gagggtataa acagtgctgg aggctggcgg ggcaggccag ctgagtcctg agcagca |

(SEQ ID NO:3;, GENBANK ™ Accession No. E13404)

TABLE 4
Mouse bone sialoprotein promoter region and cDNA

| | |
|---|---|
| 1 | tctagaaagc actgttcctt taaaatcatt caccacctct ggctcctaca atcttcctgt |
| 61 | cctcccttcc acacagatcc ctgagccttg aggagagggc tgtgataaat catccccttt |
| 121 | ggagtgagca gtctgaagtc tctcattctc catgcactgt cttattccgt cccgcgggat |
| 181 | tcagttattc gtgggtgcga gggggaccac gaacctggaa ggaaatggga ggaaaagaaa |
| 241 | gagagcggac gaccaagtag attgaacata tcaaggtctc gtttattagg ctgaggtgcc |
| 301 | ttctttttaa agcatacatc acggggaata tgggaggggt cgagggagaa ttatacaaag |
| 361 | aacaaagaag tgggcatctg ctgacatggg ggccgaagtc aggcgccagg cagcgggcac |
| 421 | tctggatttt atctctggaa cattgatcct ccttgacagc cttgggggtc aggctgggct |
| 481 | caggcgtaac tcatgtcctt ggatggcacg ggaactcagg aagagatagg gaagagggga |
| 541 | ctataattca gcttttacag cctcaggtgc caagaaagga atagggagga aggggggtga |
| 601 | taaccagctc ttagtacaag gccatttggc ctgttaggga gattgtgaag ggctcacttt |
| 661 | ctcacgggat ggtctctgac actgtctggc tgtgtgtctc cccatctact gcaagactgg |
| 721 | gcttttctga tgaagtgtaa gcctagtgag ggtgccctgt tcattagaag tcattttgca |
| 781 | gtcactcagc agaatattag tagtgggttt ctttccccct gagagctcac aacctgtcta |
| 841 | gtctcgggtt cttagcaccg tgaataattc tattttcaga agttaacatc cttcccctca |
| 901 | gacacctttg aagcttgtgg gtgtttgggt ttctgtgccc tctacctgca cgtctctcca |
| 961 | tacccaactg tgagcatttg aaagcgtgtg ctagagtttc ttgtttagct ccccatgtcc |
| 1021 | tataaaacac tttggtttgg tagagaactg agcagttcaa actttgctca actgagctta |
| 1081 | tgggggtgaa ttgaatacaa gcaaataaaa ggagcttatt caacttctct tttgtggttc |
| 1141 | tctattttat ttttaaatgc tgaaatactt ttctttagct aaatcatctg aagaatctaa |
| 1201 | cagagtcact actctggcaa caatactgga caacaatggc atttattgat ttctgtaaag |
| 1261 | tagaagtcaa cagagaagaa tatggggata aagaatatag ggataaagaa gacaaccaac |
| 1321 | cagagctccc agggtctaaa ccaccaacca gggagtacac atggagggac ccatggctcc |
| 1381 | atctgtatat gtagcagagg atggcctagt ccatcatcaa tgggatgaga ggcccttggt |
| 1441 | cccatgaagg cctgatatcc cagtgtcggg gaatttgagg gcagggagga gagagtggat |
| 1501 | gggtaggtgg gggaacaccc tcatagaagc aggagggggg gtgggatagg gggttttggg |
| 1561 | gtgtgggaat tgggaaaggg gataaacctt gaaacgtaaa taaataaaat atccaataaa |
| 1621 | aaaatcttct ggaaaagaaa agatatacaa aatacaaagg cagtttcctt tgcaaactta |
| 1681 | ggaaatgttc agtttgccaa tgcatgcagt aagtttattt tccagtaatt attcaataac |
| 1741 | catgaactgc tctctggcag tgctagtaat tattctctac tcataggaaa aaaattacat |
| 1801 | aagaagacga ctagaaataa gattatacga tgtgcagtgg cctcatttac acagcaaagg |
| 1861 | gccacatagg ggataatccc aaggacttgt tctatgaaag gttacatcag ctccttggtc |
| 1921 | tcaacctcga acgctgtaac gttcacagtc agcattgtgc tttagcaaag cttaggtaat |
| 1981 | ctgactggtt taataatatc agttttgact tacaagcctc tgaaatatgt ttcagggaga |
| 2041 | aatataaagg aatcaatatt aaactatctc ttggcatcaa ctcatttcct aattcagtac |
| 2101 | ttttagaccc atgcagtgct gtgtgaaagc cagctttcct ttctttcaac acagtgaaaa |
| 2161 | cctgtatcat tgtgaaagct taaatgctta agtcttttgc tatttatttt atttgaaatg |
| 2221 | cagtatatta ttatatatat tcagaactct aactaccatc ttctcctcac ccttcaatta |
| 2281 | aatcccacaa tgcaagcctc ttggcagaag gcccaccttt catgtttatt caactgaggc |
| 2341 | tgaatcttga aaatgtgttg aagtttggga ttctctggtg agaacccaca gcctgacgtt |
| 2401 | gtgctggcca cagctgtgat tggctgttga gaggcggaga agggtttata gtcagcaaga |
| 2461 | gcaagtgaat gagtgagtga cagccgggag aacaatccgt gccactcact cgactcgagc |
| 2521 | caaggacctg gccgaaagga aggttaaggt aatgggcaag gacctcacag ccaggtaatg |
| 2581 | ggcaaggacc tcacagccag gcacctcagt cttccctgtg tggctttggc ttggagtttg |
| 2641 | tagctgcagc atggatctta ctgcacagtg cacagtggct ctagttgaac ttttgcttgc |

(SEQ ID NO:4:; GENBANK ™ Accession No. AF071079; promoter region, nucleotides 1-2472)

TABLE 5
Rat bone sialoprotein promoter region

| | |
|---|---|
| 1 | aagcttaggg aacattcagc ctgccaacat acgcgggaag tttattttcc agtgatcctt |
| 61 | tcaatggccg tggaactgct ttctggcagt gctagtaatt cttctctcct cagagggaaa |
| 121 | gatacatagg aagaggactt agaaataagc ctgagagtat acagcgcttg atgacctcac |
| 181 | tcgcacaacg aaaggccatg tcccggatga tgccaactac tttgttcgat gagagttaaa |
| 241 | tcagcttctt ggtctgagcc tcaaatgttg tagctttcac agtcagcaca gttagcaaag |
| 301 | ccttggcagc ccggctggct ttacaatact gattctgact tacgagcctc tgaaatgcat |
| 361 | ttcagaaagg aatataaagg gatcttcact gaacacctct tgtcatcaac tcgtttccta |
| 421 | attcagtgct tttaggctcg ggcagtgctg tgtttaacag aggctagttt tcctttcttt |
| 481 | caacatagta aaaacctgta tcattgtgaa agtttaaatg cttaagtcgt ttgccattta |
| 541 | gtttatttga aatgcagtgt attattatag atattcagaa ctctaactac catcttctcc |
| 601 | tcagccttca attaaatccc acaatgcgac ctcttggcag caggcgcgcc tttcatgttt |
| 661 | attcaactga ggctgagtct tgaaaacgtg ttgtagttac ggattttctg gtgagaaccc |
| 721 | acagcctgac gtcgcaccgg ccgtgaccgt gattggctgc tgagaggaga agaagggttt |
| 781 | ataggtcagc aagagcgagt gaatgggtga gaggcagccg ggagaacaat ccgtgccact |
| 841 | cactcacttg ctctctccag ccaggactgc cgaaggtaag gtaatgggcc agcacctcac |
| 901 | agccacctgc ctcaggcttc ctgtgtggct ttggcttgga atttgtcgtt gaagcatgga |
| 961 | tcttactgct tggtgcacaa tggctctggt tgaactttag cttgctgtga aatgggacct |
| 1021 | ctgagtttag gttctttcca aagaccaggc tgggtaacgt aagcatgcag ttaaactgct |
| 1081 | tcagattggt acc |

(SEQ ID NO:5; GENBANK ™ Accession No. LO6562)

TABLE 6
Mouse dentin sialoprotein gene regulatory regions (5′ to exon 1)

| | |
|---|---|
| 1 | gaattctttt cccattggta acgtaaaaga ccactactta attgagttag cttaggctca |
| 61 | acaaacagac tttatacaac ttaacttcct tcacatttat gaaaaattaa tcagtatcgg |
| 121 | cactgagaag gcagaaacag gtagaactcc atgagtttca ggccagcctg atctacatag |
| 181 | gaattctagg acaagcaggg ctaggtagag ataccctatc tcaaaaaacc aaaacccaaa |
| 241 | aacattacgt ttaagcagat ttagttttga ccctaaatgt ttgtcttagt gaaggtccca |
| 301 | aatgctctta gcaaatgttt ctttgtgtag ttggagagtg ttgtgtgcta atacagctat |
| 361 | caagcacttc tgtttagaca ccgaagatct tcttaactct ccatcaggtc tggagagctg |
| 421 | ttcaaatctg ctattacaac caagttagga agaggaaggc aattcctgag gaaagtggca |
| 481 | ttcttaaata tgattggccc tttaagatgc tcaaagaacc aagaaccatg cagtgtaaat |
| 541 | aatagcaaag tgtttactat ggaagtgcag cttcgaggaa actcccttcc tatcactgga |
| 601 | acctgtccaa tccctaccta catgaatatg ttgtttaatt ctctcagtat aaagctctga |
| 661 | agatgctgtt gctggatagt gatttaatat ttctgatcat atgtgtttga catctttcag |
| 721 | tagtgtgaca taaaaacatg gacacatccc taagctggta cacagagact ccaattgcct |
| 781 | agtgtggagc tcataagcta gagaaatggc tcagggatca tcttgtatat ccagggctcg |
| 841 | agagaatgat gggttcaggc aagtactttt tcctttctgg aagcacagcc tgttttccta |
| 901 | ttctgtactc tatagtttac acatatagtg gagcaaagaa tgaaagctgt gtctgtggtg |
| 961 | tgtgtgtgtg tgcactctgt acttacgcat agatacctta caccatgttt cacctttgga |
| 1021 | acagctattt ttaaatttag tttgtattaa attaatagat tataaagaaa aacccaaaac |
| 1081 | ctttatgtca gtgtttagat taaatcagaa aggtttcctg aagttactgt ttataaattc |
| 1141 | ttttaaagat cccttaggca gtgtcaagac tgttgcatgc ggacagccgc ttgaattata |
| 1201 | gcgcaccaac tttaatatgt acctcaggaa tgataggggt cttaaatagc cagtcgtatt |
| 1261 | tactagagaa acctagagtt ttcttagatt gccgacctaa gcaagaggag aaatgcaggg |
| 1321 | tgacagagtc taagtggctc ttttcagata tatcacactg attatctata tttaagacac |
| 1381 | aaaacagtct tccaggagct atttaattaa gtgaaagtaa gtctagtcct tttggaacca |
| 1441 | aaggtctcag tgagccaacg taccggcgag cgagggagtg gggcgttatt acagcctcat |
| 1501 | aggcacactg actctttaaa cccccacatc agggatccta agcagtgatt ggttgagaaa |
| 1561 | attatcaaac tgaatttaaa tttcagcagg tacaaaattg tcacgcaaaa agcccaggac |
| 1621 | agtgtgc |

(SEQ ID NO:6; nucleotides 1-1627 of GENBANK ™ Accession No. AJ002141).

TABLE 7

Mouse dentin sialoprotein gene regulatory regions (intron between
exon 1 and exon 2)

| | |
|---|---|
| | gtaagat ggactccctc ctgccaggag ccaactgtct cctgttgaga |
| 1741 | gaatctccag ctgcagagat gagggtgact tgggataaag tttttaactc ttcaggtcta |
| 1801 | cactatatat taaagataat gtgtgattca ggaaggggtg ctaagccatc tgatgagacc |
| 1861 | atctgataag acgacgaatc actggggagc agaactgatt ttgccccagt atattgttga |
| 1921 | gactttatct cctataggaa aaacctaaga tgaaacaaac attctaattg tattaattaa |
| 1981 | aaaaaaacag tacctgaagg gttttatgta tagttctcta tagctctatt tttgttattt |
| 2041 | tcattcagga aaatactttt aagagctata aacctagtca aaggtgtttt acagccttgt |
| 2101 | ccttggaatg ttgggagtgt tgggatttaa caaatgagaa tcacacactg tcttcctctt |
| 2161 | cgagacagag acatggatga tgcagtgtcc aaacaccagc tcttcctgaa aaataagctg |
| 2221 | ggtttggggg tttgatttaa tcatggctct tcatgatttc aaggtctgcc tagtgtttat |
| 2281 | gattaaagct ctatggcgaa aagaattgtg gttcctccca gggctcagta tctgcctgat |
| 2341 | attaacttcc gatgttcact gactggacct aataaataaa tctccattta aacttagtat |
| 2401 | cttgactcag agtcaactta ggatctggga gcgtaatttt ctggcatgtg atgtgaagtt |
| 2461 | tctaaaagta gacgctcaaa cagttttatg tagaaaacac acagatctgt caagctgatt |
| 2521 | tttcagctcc aaatttcatg ataataggtt tagggaaaac aaagacatat tgcctcaagt |
| 2581 | tggcaaaaat tgaggtggaa atttgaatgt ggtcactttg aatggttttg atttaagaaa |
| 2641 | aaatagataa cttgtattgt aaatatcttt aaaatatttt tattcattcc ctgagaaatt |
| 2701 | tgtgtggtat gttctgattg ctctccccag atctgccttt gttctttact cacacaactt |
| 2761 | tgtgctcttt ttgtaaagaa acaaaacaag agccatgcac accagtttgt gctcctcaaa |
| 2821 | tgtactcagc tgtgtggcca tctgctgggt tctggttgcc ttaccagggg ctacattctt |
| 2881 | ggagaacact gcctttcctt ttttcccacc acctattgtt aattgttctt catgtccagc |
| 2941 | tttcctctcc ttgctgggat ttggtctgac ttgggcttgc acggtcgggt gcaggctgtc |
| 3001 | agaagcgctg tgaagatagc tcgggtagtt taagtctacc tcaggcattc caacaaggcc |
| 3061 | ctcacaatga ggctttgcgt ttcctggtct tcttagtgag tgatatattc attctaactg |
| 3121 | gctattcata catttcatct agtgtggggc aataaatggg acaatttaaa ggagcctcaa |
| 3181 | ttctaatgac tggttatttc caccagggtc tttgatatgg ttgacctgcc ttgccaacag |
| 3241 | gtgcaagtat catatatgtc agtgctggag tggaaatgtg gtgtgtgtgt gtgtgtgtgt |
| 3301 | ccgtgtgtgt gtgtgtgtgt gtgtgtgtgt aaggagggat ggaaggtgga tggtgggaga |
| 3361 | caggaattct cagatggtca gatttcagtt tagaaattat atgtgtgtgt gtgtgtgtct |
| 3421 | gtctgtctgt ctggacttta ttgcaggtac ctttccagga ccagggatcc ccagttcaca |
| 3481 | ctcggtttag agttgccaag ctcaagtata agcttggctt ggtagacaga tggccttcac |
| 3541 | ctcaactcct ggccctgggg ctttgtctca aggcacctca ttttagtttg tagaataatt |
| 3601 | gaagggaccc cagcttttct tagctttctc ttgacagcta taaggaaggg tgaagcatct |
| 3661 | ttttcagaga tcctagaatt gtgttctcac ttctgtcaag taataaacaa tatatattca |
| 3721 | ttgatgtttt attctattcc cctattaacc ttggatttta atcaaggaca ttttatgatg |
| 3781 | tgcaaggtgg taatcattaa ttcttgtgga aggtcacaag ataggagaaa acaattcttt |
| 3841 | ctatagtaaa acaccatgat acaaataaat ttagttttag aaaatgggaa cctgaagttt |
| 3901 | tgattcacat agatttttat agttttacag gctccattcc aatgtatgaa aaatatgtat |
| 3961 | ctgattctgt gaatttgcat tgcaaagggt gaaagatttc actcttgaag cctctctcct |
| 4021 | tcagctcctc cctcagtccg agactgcata gtgcccgggt aagggtgggg tgtcctttgt |
| 4081 | cctcaggagt gcttgttcag cagcaggctc tgcaaggtga cctttgcttt gctcagaaga |
| 4141 | cactgatgat caagatgctg gcgtgggctc cgagacctga tgccagtgag gaggaagatg |
| 4201 | gggtagctag gcaacttcaa aacagtgcaa tgtgctgcca gcatcgagcg agcggagggt |
| 4261 | gcacaagctg atgctgtgtg aggaagggag ctaaagatgc cttcagaaag ctttttgggg |
| 4321 | gtgattcttc tgccaacccc taggatattg tgagctacag agttattaaa ccagactgag |
| 4381 | gaaacaaaag cccaataaag ctattgaaag tgcccaagct cagagagcag atagcagggg |
| 4441 | aaggatttga attcagggat ctgaaaccaa atcctgtgtt ctctctccta gcctaaactc |
| 4501 | tctcttcctt aaacactgta agaggaagat ttcttcctct tactgggata acgcccaatt |
| 4561 | ctatatagac caggtgggaa attacaagtg ctttatcatt tacaatctac ttttagttaa |
| 4621 | tgatgcttaa agctagccca ggagagacgt taccctcatg gataacagca tagggccaga |
| 4681 | gccacgagct atgtactctg tatcttcatg gctgttgctt ccacaggcag gtagagtcag |
| 4741 | aagccatgac agtcctgagc atgcagaggc ccccacatac ccaggtttat ttctggaacc |
| 4801 | tggggtgttt tctcacatta gtactttctc cttgtcctag aaaagggcca aatgtaagac |
| 4861 | caaaatattg gggtactgtg gctgtcatct ttcatcttat gacccgtttt gtggtgttct |
| 4921 | ttgttctaaa cag |

(SEQ ID NO:7; nucleotides 1-1627 of GENBANK ™ Accession No. AJ002141)

Expression of the nucleic acid is preferably inducible. Osteoblast or odontoblast transcriptional regulatory DNA is used to control expression of IL-4 or another anti-inflammatory polypeptide in a transcription unit. A truncated fragment of such promoters, e.g., containing part of SEQ ID NOS:3, 4, 5, 6, or 7, which functions to preferentially direct transcription in odontoprogenitor cells or OPCs (compared to other cell types) may be used. The regulatory sequence, e.g., a cis-acting cell-specific transcriptional regulatory element, is positioned 5′ to a heterologous nucleic acid sequence, in a transcription unit. All or part of one of the nucleotide sequences specified in a reference sequence, its complementary strand or a variant thereof may be used in to direct transcription of a heterologous nucleic acid sequence. A nucleic acid fragment is a portion of at least 20 continuous nucleotides identical to a portion of length equivalent to one of the reference nucleotide sequences or to its complement.

Expression of a heterologous polypeptide-encoding sequence is regulated by contacting the cells of the invention with an antibiotic compound such as tetracycline or a tetracycline analogue (e.g., minocycline or doxycycline). For example, tetracycline is systemically administered at least 2 days before periodontal surgery, e.g., and the time at which cells of the invention are implanted, and/or for at least 2 days after surgery and/or implantation. Expression of the heterologous polypeptide by the cells is turned on while the antibiotic is present in the tissue, i.e., while it is being administered to the cell implant recipient. Expression of the recombinant anti-inflammatory polypeptide decreases and ceases after administration of the antibiotic is stopped. Typically, an antibiotic administered 8-12 days prior to surgery and 8-12 days post-surgery. Similarly, antibiotics are administered before and after orthopedic surgery, e.g., surgery for cartilage removal from articulating joints or for removal of metastatic bone tumors (at which time the cells are implanted at or adjacent site to diseased tissue). The cells may be implanted before, during, or after implantation of a dental orthopedic prosthesis. To treat advanced periodontal disease, the cells are administered locally to the periodontal ligament in the mandibular section of the jaw. A clinical benefit is conferred by using the cells to inhibit osteolysis in a mammal, e.g. a human patient, that is suffering from or at risk of developing periodontitis or other bone disorders which may lead to bone loss, e.g., alveolar bone loss.

The methods described herein are also applicable to veterinary use, e.g., to treat dogs, cats, horses.

The invention includes OPCs which are genetically modified to contain a nucleic acid encoding an anti-inflammatory polypeptide. OPCs are derived from bone marrow stromal cells and have been differentiated ex vivo in the presence of ECM. As is described above, the OPCs preferably contain a nucleic acid encoding a cytokine such as IL-4, or an agonist thereof, operably linked to a promoter which directs transcription of a nucleic acid to which it is linked preferentially in cells which have differentiated into osteoblasts.

For treatment of bone disorders, the cells are implanted into the bone marrow of a recipient mammal or into an articulating joint of the mammal. For example, the cells are administered intratibially or intrafemorally. The cells are implanted locally, e.g., at the site of bone loss or adjacent to such as site, e.g, in the bone marrow, and expression of the recombinant polypeptide by the cells is regulated by systemically administering an antibiotic such as minocycline or doxycycline. Methods of transplanting cells into the bone marrow of a mammal are well known in the art, e.g., as describd in U.S. Pat. No. 4,188,486. The dose of cells to be administered ranges from 1×10 cells to 1×10¹⁰ cells in volume suitable for the location of transplantation (e.g., a smaller volume is used for implantation into mandibular tissue or into the periodontal ligament compared to implantation into the bone marrow of the femur). Clinical protocols for such implantation procedures are known in the art. For example, a dose of 1×10⁸ cells per kg of body weight is administered to femoral bone marrow. Repeated implants may be required in the case of long term diseases such as rheumatoid arthritis.

Inhibitors of cyclooxygenase II (COX-2) or tumor necrosis factor-alpha (TNFa) are optionally administered. COX inhibitors include aspirin, ibuprofen and indomethacin, as well as bisaryl COX-2 inhibitory compounds (e.g., as described in U.S. Pat. No. 5,994,379) and (methylsulfonyl)phenyl-2-(5H)-furanones (e.g., as described in U.S. Pat. No. 6,020,343).

The isolated genetically-modified OPCs are used to treat individuals suffering from or at risk of developing a bone loss disorder such as rheumatoid arthritis, osteoporosis, periapical or endochondral bone loss, artificial joint particle-induced osteolysis, bone fracture or deficiency, primary or secondary hyperparathyroidism, metastatic bone disease, osteolytic bone disease, post-plastic surgery, post-prosthetic joint surgery.

By the term “osteoprogenitor” is meant a differentiated bone precursor cell derived from a bone stromal cell. By the term “odontoprogenitor” is meant a differentiated bone precursor cell derived from periodontal ligament. The differentiated state of the bone marrow stromal cells or ligament derived cells is induced by culture in the presence of ECM. Preferably, the cells are cultured in the presence of a BMP such as BMP-2, 4, or 6. Differentiated progenitor cells have enhanced ability to build bone tissue, compared to undifferentiated stromal cells. OPCs or odontoprogenitor cells are distinguished from bone stromal cells (as well as fat, muscle, or cartilage cells or tissue) by the production of alkaline phosphatase, expression of osteocalcin, and expression of bone sialoprotein (in addition to the expression of dentin sialoprotein in the case of odontoprogenitors).

The ex vivo cell-based therapeutic methods of the invention has several advantages over standard gene therapy protocols. For example, the cells expressing the recombinant anti-inflammatory polypeptide are isolated, i.e., purified from cells which do not have the desired phenotype. A population of isolated OPCs or odontoprogenitor cells is at least 75%, more preferably 85%, more preferably 90%, more preferably 95%, more preferably 98%, more preferably 99% or 100% OPCs or odontoprogenitor cells, respectively.

DNA is introduced into isolated cells ex vivo, thus avoiding or minimizing the possibility DNA uptake by non-target cells in the body. Another measure of safety is conferred by using a transcriptional regulatory element and a promoter that directs transcription only in the isolated cell type. In vivo expression of the recombinant polypeptide is further regulated by the systemic administration of an antibiotic or antibiotic analogue.

OPCs are isolated and expanded from stromal cells from bone marrow aspirates, and autologous bone marrow stromal cells are expanded. The cells are optionally frozen and stored in liquid nitrogen for long periods of time before being differentiated and transduced. These “banked” autologous cells allow for multiple inoculations over a long period of time, which is advantageous since RA may persist for many years

Other features and advantages of the invention will be apparent from the following detailed description and from the claims.

### BRIEF DESCRIPTION OF THE DRAWINGS

FIG. 1 is a bar graph showing alkaline phosphatase acitivity of rabbit bone marrow stromal cells plated on ECM produced by untransduced C3H10t1/2 cells, or C₃H₁₀T1/2 cells transduced with and without BMP-6, or on plastic alone. Activity is expressed as μmol p-nitrophenol produced/min/mg protein×10⁻⁴ at days 1 and 21.

FIG. 2A is a diagram of a periodontal ligament biopsy.

FIG. 2B is a diagram of the method for differentiating odontoblast precursor cells on an osteoinductive matrix and transduction of the cells with a regulatable therapeutic gene.

FIG. 2C is a diagram of a device for implanting cells adjacent to teeth in need of therapeutic intervention.

FIG. 3A is a photomicrograph of rabbit bone marrow stromal cells cultured for 21 days after plating on tissue culture plastic. Magnification, 100×.

FIG. 3B is a photomicrograph of rabbit bone marrow stromal cells cultured for 21 days after plating on ECM-coated dishes from a C3H10T1/2 cell line transduced with rhuBMP-6. Magnification, 100×.

FIG. 4 is a bar graph showing the effect of rhuIL-4 on rhuIL-1 alpha-induced PGE2 by rabbit osteoprogenitor cells.

### DETAILED DESCRIPTION

Cell-based delivery of rhIL-4 at the site of an osteolytic lesion allows rhIL-4 to be concentrated near an inflammatory site where inflammatory effector cells, e.g., macrophages, and osteolytic effector cells, e,g., osteoclast precursor cells, are located. Adverse effects of rhIL-4 on thymocyte and T cell function are greatly decreased since the cytokine acts locally as opposed to functioning throughout the body when delivered systemically.

The cells described herein are committed to the osteoblastic lineage. Differentiation was induced by stimulating the bone marrow stromal cells to differentiate by exposing the cells to an extracellular matrix such as Matrigel (Becton Dickenson) or other commercially available matrix preparation in the presence of bone morphogenetic proteins. This step is typically carried out before the differentiated cells are transduced with retroviral expression vectors containing genes encoding one or more therapeutic proteins. This is advantageous in that the transduced cells cannot differentiate into cartilage, muscle or fat cells once implanted in vivo. In contrast, the pre-osteoblastic pluripotent bone marrow stromal cells still retain the potential to differentiate.

Unlike the retroviral vectors employed in the prior art which use viral promoters, the retroviral expression vectors in the OPCs have been constructed to use osteoblast specific promoters to initiate transcription of the reverse transactivator Tetracycline Activator (rtTA) gene, which in turn regulates production of the rhIL-4 therapeutic protein. This approach provides the advantage of increased safety because the osteoblast promoters direct transcription more efficiently in OPCs (compared to other cell types such as stromal cells) and are far less likely to be inactivated in vivo than viral promoters. For example, the viral vectors contain a doxycycline-inducible system which regulates the expression of the interleukin-4 encoding sequences.

The OPCs described herein were modified to increase the expression of the alpha-5 integrin receptor. This modification allows the cells to adhere to bone matrix proteins when implanted in vivo, which confers the added advantage that the OPCs may be inserted directly into osteolytic sites without prior encapsulation, e.g., in porous calcium phosphate ceramic cubes or other types of encapsulated devices.

The cells are used for autologous or allogeneic cell transplants to serve as a cell-based platform to deliver the recombinant human interleukin-4 (rhIL-4) protein in a site-specific, regulated manner. RhIL-4 acts on defined cell types when the OPCs are implanted at the site of osteolytic bone lesions. The OPCs, genetically-engineered to produce rhIL-4 are used to i) promote tumor reduction when used in conjunction with anti-cancer drugs, ii) inhibit formation of osteoclasts which resorb bone, and iii) stimulate new bone growth. The methods result in improved clinical outcomes. The OPCs, engineered to secrete rhIL-4 may are implanted in patients who are undergoing revision of a artificial joint replacement due to the development of implant-induced osteolysis, as well as in patients suffering bone loss due to rheumatoid arthritis and in the oral cavity due to severe periodontal disease.

IL-4 and Agonists Thereof

IL-4, an anti-inflammatory cytokine produced primarily by Th2 cells and macrophages exhibits anti-inflammatory and immunosuppressive properties. Bone-derived cells, e.g., differentiated osteoprogenitor cells or odontoprogenitor cells, which are genetically-altered to produce recombinant human interlukin-4 (rhuIL-4) are administered to diseased bone tissue. The invention provides a gene therapy approach to deliver rhuIL-4 locally at inflammed joints by targeting bone marrow stromal cells that have undergone partial differentiation with a viral plasmid expression system containing a cell-specific promoter. In order to prevent potentially harmful effects of rhuIL-4, due to high local or systemic concentrations, the production of rhuIL-4 is regulated by the oral administration of antibiotic analogues. Articular cartilage degradation and bone resorption, associated with rheumatoid arthritis, is reduced significantly by the local, regulated release of IL-4 near the site of tissue damage. The mechanism for reduced cartilage degradation and bone loss is based on the ability of IL-4 to inhibit TNF-alpha, IL-1, and PGE2 production, as well as the ability of IL-4 to decrease angiogenesis. Regulated, local release of IL-4 decreases cartilage and bone destruction in vivo. IL-4 also has immunosuppressive properties; in situations in which the implanted cells are allogeneic (rather than autologous), the IL-4 produced by the implanted cells may obviate the need to administer systemic immunosuppressive drugs to combat tissue rejection.

### EXAMPLE 1

### Differentiation of Rabbit Bone Marrow Stromal Cells on a BMP-6 Containing Osteoinductive Extracellular Matrix Derived from C3H10T1/2 Cells

Over 185,000 spinal arthrodeses are perfomed in the US each year, with non-union rates as high as 35% reported in the most commonly performed procedure, posterolateral lumbar interansverse process fusions. Autolgous iliac crest bone is the gold standard graft material, but it is limited in quantity, and the morbidity of harvest is not insignificant. The data described herein indicate that BMPs induce differentiation of multipotential stromal cells from rabbit bone marrow an osteoblastic linage. The stromal cells are exposed to ECM secreted by a transfected murine cell line constitutively expressing or overexpressing mRNA for BMP-6.

Cells from the C₃H₁₀T1/2 murine fibroblast line were transduced with either an LXSN vector containing the rhBMP-6 gene or the same vector without the gene. The cells were cultured under standard conditions in DMEM (Gibco; Gaithersburg, Md.) supplemented with 100 units/ml PCN, 100 μg/ml streptomycin, and 10% FBS (Hyclone; Logan, Utah). Four days after reaching confluence, the cells were lysed sequentially with water and 0.1% Triton X-100. The plates were gently washed with phosphate buffered saline (PBS), leaving the extracellular matrix and the BMP-6 protein adherent to the plastic dish.

Bone marrow was aspirated from the femurs of two New Zealnad white rabbits and suspended in DMEM with 100 u heparin/cc. The cell suspension was diluted with PBS, 2% BSA, 0.6% sodium citrate, and 1% penicillin/streptomycin. The suspension was then layered on a Ficoll-Paque (Amersham Pharmacia Biotech; Piscataway, N.J.) gradient, and centrifuged at 600×g for 20 minutes. The cells at the interface were isolated, washed, and re-centrifuged at 500×g twice. They were then cultured to confluence in a T-75 flask under standard conditions in a-MEM with L-glutamine 2 mM, without nucleosides, supplemented by 12.5% FBS, 0.2 mM I-inositol, 20 nM folic acid, 0.1 mM beta-mercaptoethanol, and 1% penicillin/streptomycin. The cells were re-plated in triplicate on the ECM produced by untransuced cells, LXSN transduced cells, or LXSN-BMP6 transduced cells, or on plastic, and cultured for 21 days.

Alkaline Phosphatase (ALP) activity was determined on day 1 and 21 after plating of stromal cells on ECM. The plates were scraped and rinsed with 0.5M CAPS, pH 10.5, and sonicated. 0.5 ml of 0.5% p-nitrophenyl phosphate was added to each sonicate, and incubated at 37° C. for 30 minutes. 0.2M NaOH was added to stop the reaction, and the amount of p-nitrophenol produced was determined by spectrophotmetry at 405 nm. ALP activity was expressed as μmol p-nitrophenol/min/mg protein. Protein content was determined by Bradford protein assay (Bio-Rad, Hercules, Calif.). As an additional control, the ECM produced by each C3H10T1/3 cell line was also assayed for ALP activity at day one, and after exposure to media for 21 days.

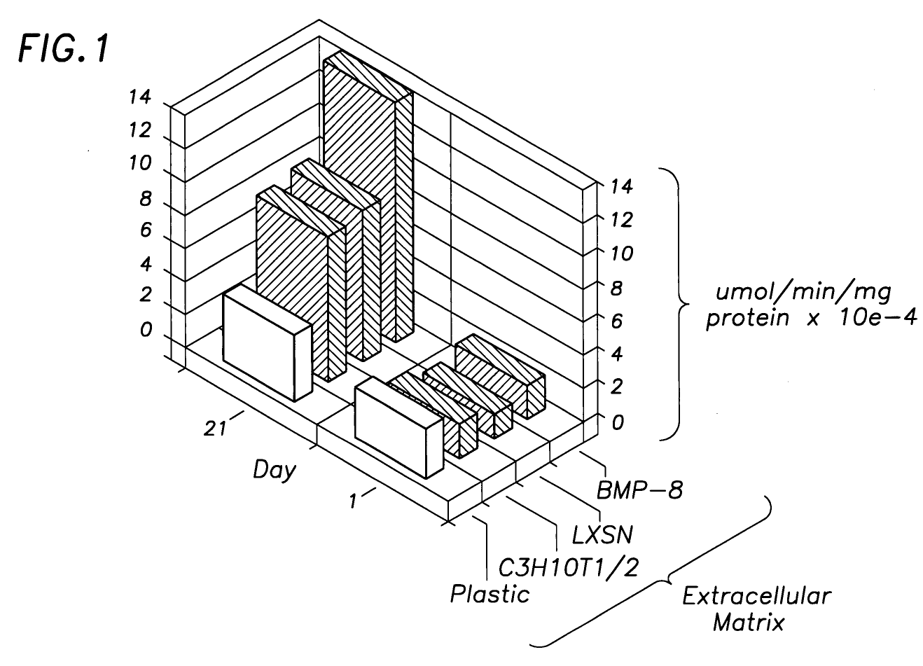

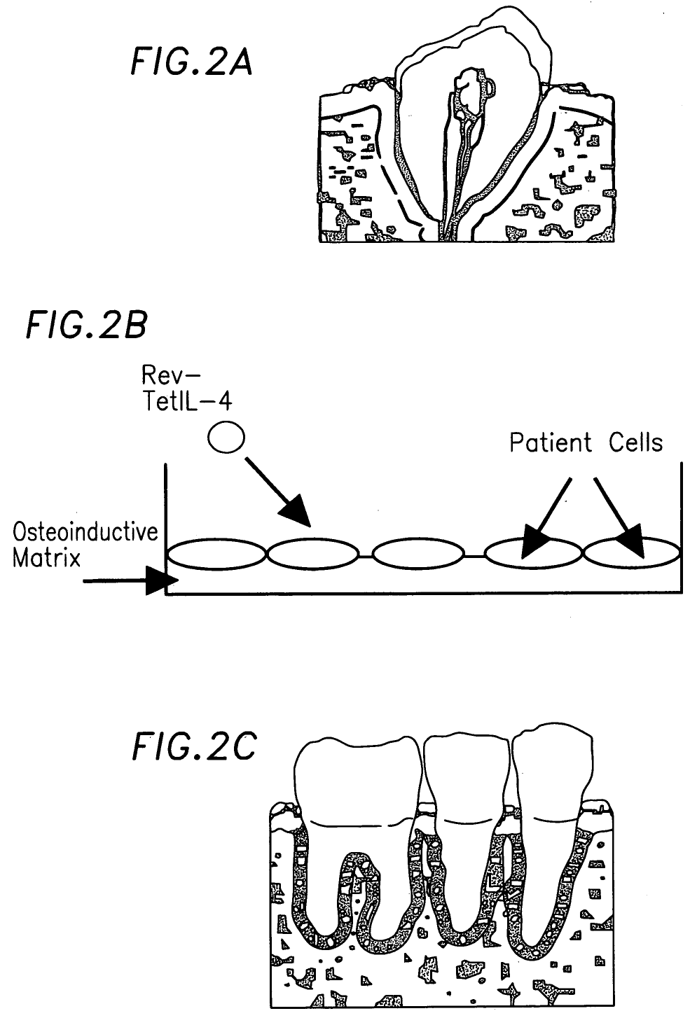

The ALP activity in the ECM alone was negligible at day 1 and 21. The activity from each of the ECM-exposed marrow cells was likewise negligible at day 1, as was the activity from the cells on plastic. However, at day 21, there were striking differenced (FIG. 1). The ALP level produced by marrow cells plated on plastic was unchanged. That of marrow cells plated on the ECM from untransduced or LXSN cells ech increased 400%, while that of the marrow cells plated on the ECM from BMP-6 transfected cells increased 700%.

ECM-bound BMP 2 and 4 produced by neonatal mouse calvarial cells stimulated ALP activity in mouse bone marrow cells. Exposure of stromal cells to EMC in the absence of BMP-6 increased ALP production, presumably due to the presence of type collagen in the matrix. The further increase of ALP production by BMP exposed cells is due to increased osteoblastic differentiation of the stromal cells.

These results indicate that exposure to ECM-bound BMPs induces stromal cells to differentiate along an osteoblastic lineage. Cells of the osteoblastic lineage, e.g., OPC's or odontoprogenitor cells, are identified and purified by virtue of their expression of marker genes such as alkaline phosphatase, osteocalcin, and bone sialoprotein (in addition to dentin sialoprotein in the case of odontoprogenitors). Probes to detect the marker genes are known in the art (e.g., as described by Guo et al., 2000, Calcified Tissue International 66:212-216). Marker gene expression is detected by measuring transcription of the genes (e.g., using labeled nucleic acid probes in in situ assays) or by immunohistochemistry to detect antibody binding to the gene products. The assays described above are used to distinguish stromal cells from OPCs and odontoprogenitor cells.

### EXAMPLE 2

### Inhibition of Alveolar Bone Loss by Cell-Delivered IL-4

The osteoclast is responsible for mediating excessive bone resorption during progressive periodontitis. IL-4 inhibits osteoclast differentiation and function. Autologous cells are engineered to express IL-4 and permanently implanted at sites of inflammation, e.g., in the mandible, in soft tissue adjacent to affected teeth, or in the periodontal ligament, using methods known in the art.

Periodontal disease is induced in C3H mice by repeated injections of LPS derived from the clinically-relevant microorganism Porphyromonas gingivalis, an art recognized model of periodontal disease. Mice with periodontal disease are treated using C3H10T1/2 cells genetically engineered to produce IL-4 in a regulatable manner. Production of interleukin-4 is regulated by providing antibiotic orally, e.g., in the drinking water. Cells are implanted locally, at sites of bone resorption, thereby bypassing the need for either systemic administration or repetitive local injections of a bioactive molecule. Optionally, antibiotics are placed in the periodontal pocket following implantation of cells for periodontal disease. This cell-based approach for local delivery of interleukin-4 utilizes tissue engineering to inhibit resorption of alveolar bone.

The murine molony retroviral vectors used herein are well characterized and are non-immunogenic in humans or mice.

Standard in situ hybridization (ISH) is used to detect IL-4 production as well as characterize the osteoclast phenotype in cells that have populated mandibler bone or other bone tissue of cell implant recipients.

### EXAMPLE 3

### Gene Therapy Vector

A tetracycline analog-regulated expression system is used to direct production of recombinant anti-inflammatory compositions. A dual “tet-on” retroviral system is used for the following reasons; i) the vectors are commercially available and the packaging cells produce high retroviral titers, ii) the murine molony retroviral vectors have been well characterized, are non-immunogenic, and have been used in safely in humans, and iii) the use of two retroviral vectors in the “tet-on” mode prevents “leaking”, i.e., recombinant polypeptide expression is extremely low or absent without antibiotic present.

“Tet-off” and tet-on” systems use the antibiotic tetracycline or various analogues to regulate expression. Toxicity of the VP 16 viral transactivator fusion protein (tTA) was not observed and no antibodies were made to the “reverse” (rtTA, tet-on) or rTA, “tet-off” transactivator. The tetracycline analogue doxycycline is the prefered antibiotic inducer for the “tet-on” system and is administered by orally or by intraperitoneal administration using known methods, e.g., as described for mifepristone and rapamycin, as well as by implantation of subcutaneous pellets. Doxycycline and/or minocycline is given orally. Minocycline inhibits the action of matrix metalloproteinases (MMPs) which are involved in breakdown of bone and cartilage. Minocycline-activated site-specific IL-4 production at the inflammed joint acts in a synergistic manner to inhibit inflammation and angiogenesis. These two drugs may work together and lead to increased benefit for patients suffering from RA. The ability to shut down local IL-4 production by removal of minocycline in the “tet on” gene system is advantageous to prevent deleterious effects of sustained IL-4 production at a site of inflammation such as in a rheumatic joint.

Some viral promoters/enhancers used in adenoviral and retroviral plasmid vectors are inactivated by interferon-gamma and tumor necrosis factor-alpha in vivo. These include the rous sarcoma virus (RSV), simian virus 40 (SV-40), and cytomegalo-virus (CMV) promoters. Since levels of IFN and TNF are elevated in RA and OA patients, the use of these standard viral vectors could limit recombinant polypeptide expression, especially if sustained production is required. Given these limitations of viral promoters for long term in vivo use, the invention utilizes a constitutive cellular promoter in place of the CMV promoter to control expression of the rtTA transactivator in one of the two retroviral plasmid vectors. A human osteocalcin promoter sequence (e.g., SEQ ID NO:3) is employed to modify a “tet on” retroviral vector for transduction of OPCs ex vivo.

Late-stage rabbit osteoprogenitor cells obtained from bone marrow stromal cells were isolated and characterized using marker gene detection. The rabbit OPCs have undergone partial differentiation on a osteoinductive matrix derived from C3H10T1/2 cells that have been transduced with a retroviral vector expressing recombinant human bone morphogenetic protein-6 (rhuBMP-6).

Bone marrow stromal cells are obtained from an individual such as a human patient 8-12 weeks prior to therapy. The cells are expanded, differentiated and transduced with recombinant DNA encoding anti-inflammatory polypeptides ex vivo. The OPCs are implanted in the marrow bones adjacent to the diseased or injured site, e.g., in the mandible or periodontal ligament for periodontal disease or in the marrow of the distal femur and proximal tibia, i.e., in juxtaposition to an inflammed knee joint. This approach allows the rhuIL-4-transduced OPCs to be in close proximity to the bone-resorbing osteoclasts and the pannus/bone interface of the joint. Correct positioning of the implanted OPCs in the marrow is important for the following reasons; i) inhibition of bone resorption by IL-4 is optimized by close proximity of the OPCs to the synovial fibroblasts of the invading pannus and the surrounding osteoblasts, ii) the locally produced IL-4 inhibits osteoclast formation from the differentiated synovial marrow-derived macrophages in the presence of rheumatoid synovial fibroblasts, and iii) the OPC-produced IL-4 locally inhibits neovascularization of the inflammed joint.

Other advantages of using bone marrow stromal cells, which have been partially-differentiated toward the osteoblastic lineage, include a high level of expression of the rhuIL-4 for a given cell population due to the higher number of osteoblastic cells locally present. Moreover, a pure population of differentiated stromal cells is not required due to the high specificity of the osteocalcin promoter for osteoblasts. Adipocyte, muscle, and chondrocytic precursor cells will not express the rhuIL-4 even if these cell types take up the recombinant anti-inflammatory polypeptide-encoding DNA. These features provide a safer and more controlled environment for cytokine release when the transduced osteoblast cells are inoculated into the bone marrow.

### EXAMPLE 4

### Construction of the rhIL-4 “Tet-On” Dual Retroviral Expression Vectors

The pRevTet-On and pRev-TRE retroviral expression vectors are publically available from Clontech Laboratories, Inc. (Palo Alto, Calif.). Both vectors were derived from pLNCX, a retroviral vector capable of producing high-titer virus in the Retropak™ (Clontech, Inc.) PT67 packaging cell line. The core murine Moloney leukemia viral vector for each plasmid consists of a 5′ long terminal repeat (LTR) containing a promotor (L) which drives the extended retroviral packaging signal. The remaining DNA consists of pBR322-based plasmid sequences which allow for replication in bacteria and an ampicillin resistance gene for bacterial selection. All plasmids and retroviral expression vectors were purified by cesium chloride/ethidium bromide ultracentrifugation gradients, checked for purity on agarose gels, and analyzed for orientation by restriction enzyme mapping and DNA sequence analysis.

pRev-Tet-On-huOC Plasmid Construct

The pRevTet-On vector also contained a neomycin phosphotransferase gene, then an internal minimal immediate early cytomegaloviral (CMV) promoter which drives the reverse tetracycline regulatory element (rtTA). A 1.339 kb BamHI/EcoRI cDNA was excised from the pGoscas vector which contains a cDNA of the human osteocalcin promoter. The regulatory DNA was ligated into pRevTet-On digested with BamHI/ClaI, which removes the CMV promoter and the rtTA portion of the vector. A 1.05 kb cDNA to rtTA was subsequently ligated into the ClaI site of pRev-Tet-On followed by blunt end ligation. This resulted in substitution of the viral CMV promoter/enhancer for the osteoblast specific human osteocalcin promoter in the pRev-Tet-On retroviral vector.

pRev-TRE-rhuIL-4 Plasmid Construct

The pRev-TRE vector (Clontech), contains a 5′ long terminal repeat (LTR) containing a promotor which drives the extended retroviral packaging signal. The transactivator response element (TRE) contains seven direct repeats of the tetO operator sequence upstream of a minimal CMV promoter, which can be bound by the tTA and rtTA transactivators. The rhuIL-4-pCD plasmid was obtained from American Type Culture Collection (ATCC (#57593). A 0.86 kb BamHI hIL-4 insert was isolated and used for subsequent subcloning into the BamHI site of the multiple cloning site in the pRev-TRE vector. Restriction enzyme mapping was performed to check for the correct orientation of the rhuIL-4 cDNA insert. The pRev-TRE-rhuIL-4 vector was subsequently transfected into the PT67 packaging cell line (Clontech), selected with Hygromycin B, and high titer clones were assayed using serial dilutions of viral supernatants before infection and Hygro B selection of NIH3T3 cells. PT67 packaging cells containing the pRev-TRE-rhuIL-4 vector were used to sequentially infect the rabbit osteoprogenitor cells along with the pRevTet-On-huOC vector. The transduced cells are subjected to selection with G418 and Hygromycin B and clones producing rhuIL-4 in response to tetracycline ana-log treatment are isolated. The optimal doxycycline and minocycline concentrations for induction of rhuIL-4 production in the transduced cells is performed using standard methods.

### EXAMPLE 5

### Isolation and Osteogenic Differentiation of Rabbit Bone Marrow Stromal Cells

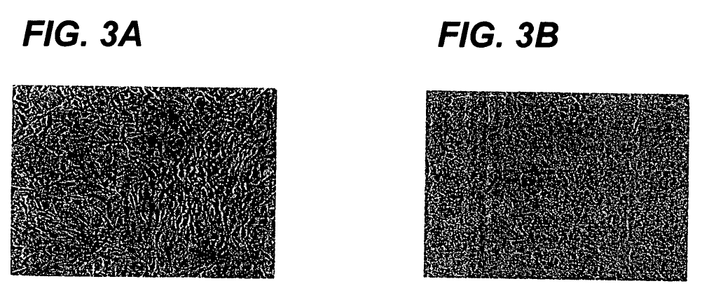

Bone marrow aspirate (approximately 1-2 c.c.) was obtained from the greater trocular region of the femur of anesthetized New Zealand white rabbits and suspended in DMEM with 100 u heparin/cc. The cell suspension was diluted with PBS, 2% BSA, 0.6% sodium citrate, and 1% penicillin/strep-tomycin. The suspension was then layered on a Ficoll-Paque (Amersham Pharmacia Biotech; Piscataway, N.J.) gradient, and centrifuged at 600×g for 20 minutes. The cells at the interface were isolated, washed, and recentrifuged at 500×g twice. They were then cultured for 2-3 weeks until confluent in T-75 flasks at 37 degrees C. in 5% CO₂ in alpha-MEM with L-glutamine 2 mM, without nucleosides or glucocorticoids, supplemented by 12.5% horse serum (Sigma; St. Louis, Mo.), 12.5% FBS, 0.2 mM I-inositol, 20 nM folic acid, 0.1 mM B-mercaptoethanol, and 1% penicillin/streptomycin. The cells were then harvested in DPBS/EDTA/pancreatin and stored in liquid nitrogen in freezing media Aliquots of BMSCs were subsequently thawed and replated in triplicate on plastic dishes or dishes coated with the ECM produced by untransduced cells, LXSN transduced cells, or rhuBMP6 transduced cells, and cultured for 1 and 21 days. Examination of the thawed cells by phase contrast microscopy revealed that BMSCs cultured on tissue culture plastic for 21 days retained their fibroblastic, spindle-shaped morphology, as shown in FIG. 3A. In contrast, BMSCs plated on the BMP-6-containing ECM for 21 days became more cobblestone-shaped in appearance (FIG. 3B) and ressembled osteoblast cells.

Alkaline phosphatase (ALP) activity was determined on day 1 and 21 after plating of stromal cells on ECM. Representative results are shown in FIG. 1. These data indicate that stromal cells may be isolated, expanded, and stored frozen before undergoing further differentiation ex vivo. The data also indicate that BMP-responsiveness with respect to alkaline phosphatase induction demonstrates that the BMSCs have undergone partial differentiation to become late stage osteoprogenitor cells

### EXAMPLE 6

### Effects of rhuIL-4 on Rabbit Late-Stage Osteoprogenitor Cells

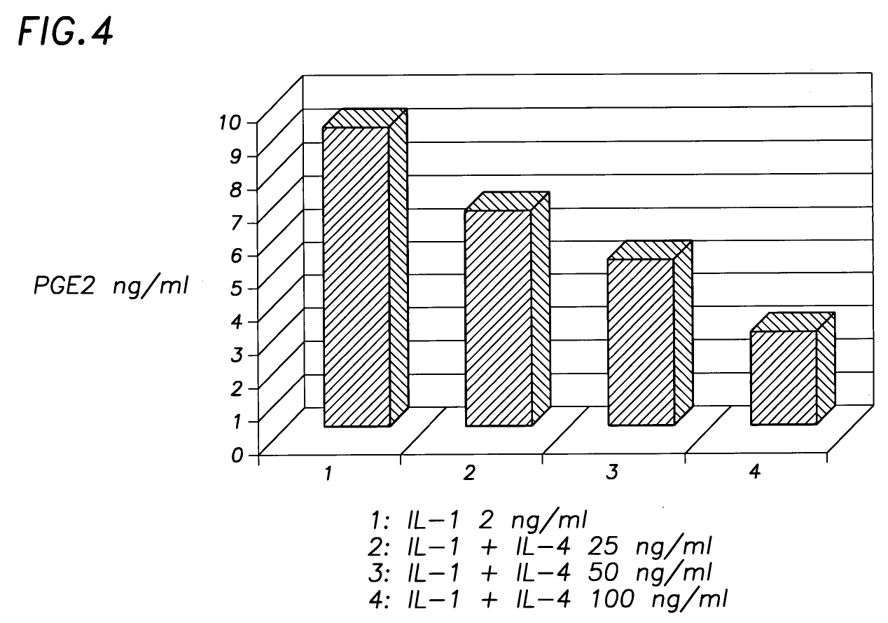

The effect of rhuIL-4 on PGE2 production by late-stage rabbit OPCs was analyzed. OPCs were harvested from the BMP-6/ECM coated dishes by trypsinization, counted, then replated in 6-well dishes in alpha-MEM plus 1% FBS and 10⁻⁵ M arachidonic acid for 24 hr. The cells were then incubated for an additional 24 hr with rhuIL-1 (2 ng/ml) in the absence or presence of IL-4 (25, 50, and 100 ng/ml). PGE2 levels were measured in the conditioned cell cul-ture media by enzyme immunoassay (BioTrak RPN 222, Amersham Pharmacia Biotech, Inc., Piscataway, N.J.). As shown in FIG. 4, there was dose-related effect of IL-4 on the innibition of IL-1-stimulated PGE2 release by the rabbit OPCs.

These results indicate that rhuIL-4 blocks the effect of IL-1-alpha on the induction of PGE2 production in osteoprogenitor cells, an important intermediate step in osteoclast-mediated bone resorption and that IL-4 is beneficial for reducing inflammation associated with RA (since PGE2 is a potent mediator of the pain and edema asso-ciated with rheumatoid synovitis).

What is claimed is:

1. An isolated odontoprogenitor cell comprising a nucleic acid encoding an anti-inflammatory polypeptide

2. The cell of claim 1, wherein said cell is derived from a periodontal ligament.

3. The cell of claim 1, wherein said polypeptide is a cytokine.

4. The cell of claim 3, wherein said cytokine is interleukin-4 (IL-4).

5. The cell of claim 1, wherein said nucleic acid is operably linked to an osteoblast-specific promoter.

6. The cell of claim 5, wherein said osteoblast-specific promoter is an osteocalcin promoter.

7. The cell of claim 5, wherein said osteoblast-specific promoter is a bone sialoprotein promoter.

8. The cell of claim 1, wherein expression of said nucleic acid is inducible.

9. The cell of claim 1, wherein expression of said nucleic acid is regulated by an antibiotic compound.

10. The cell of claim 9, wherein said antibiotic compound is tetracycline or a tetracycline analogue.

11. The cell of claim 10, wherein said tetracycline analogue is minocycline or doxycycline.

12. A method of inhibiting osteolysis in a mammal, comprising introducing into said mammal an isolated odontoprogenitor cell comprising a nucleic acid encoding an anti-inflammatory polypeptide.

13. The method of claim 12, wherein said mammal is suffering from or at risk of developing periodontitis.

14. The method of claim 12, wherein said mammal is suffering from or at risk of developing alveolar bone loss due to periodontal disease.

15. The method of claim 12, wherein said cell is administered to the periodontal ligament in the mandibular section of the jaw.

16. An isolated osteoprogenitor cell comprising a nucleic acid encoding an anti-inflammatory polypeptide.

17. The cell of claim 16, wherein said polypeptide is a cytokine.

18. The cell of claim 17, wherein said cytokine is interleukin-4 (IL-4).

19. The cell of claim 16, wherein said nucleic acid is operably linked to an osteoblast-specific promoter.

20. The cell of claim 19, wherein said osteoblast-specific promoter is an osteocalcin promoter.

21. The cell of claim 19, wherein said osteoblast-specific promoter is an bone sialoprotein promoter.

22. The cell of claim 16, wherein expression of said nucleic acid is inducible.

23. The cell of claim 16, wherein expression of said nucleic acid is regulated by an antibiotic compound.

24. The cell of claim 23, wherein said antibiotic compound is tetracycline or a tetracycline analogue.

25. The cell of claim 24, wherein said tetracycline analogue is minocycline or doxycycline.

26. A method of inhibiting osteolysis in a mammal, comprising introducing into said mammal an isolated osteoprogenitor cell comprising a nucleic acid encoding an anti-inflammatory polypeptide.

27. The method of claim 26, wherein said cell is implanted into an articulating joint of said mammal.

28. The method of claim 26, wherein said cell is administered intratibially.

29. The method of claim 26, wherein said cell is administered intrafemorally.

30. The method of claim 26, wherein expression of said polypeptide is regulated by an antibiotic compound.

31. The method of claim 26, wherein said antibiotic compound is tetracycline or a tetracycline analogue.

32. The method of claim 31, further comprising administering minocycline to said mammal.

33. The method of claim 30, wherein said antibiotic compound is administered systemically.

34. The method of claim 26, further comprising administering an inhibitor of cyclooxygenase II (COX-2).

35. The method of claim 26, further comprising administering an inhibitor of tumor necrosis factor-alpha (TNFα).

36. The method of claim 26, wherein said mammal is suffering from or at risk of developing rheumatoid arthritis.

37. The method of claim 26, wherein said mammal is suffering from or at risk of developing periapical or endochondral bone loss, artificial joint particle-induced osteolysis, or osteolytic bone metastases.

38. A method of inducing differentiation of a bone marrow stromal cell, comprising contacting said cell with bone morphogenic protein-6.


SEQUENCE LISTING (7 sequences)

SEQ ID NO: 1 - protein, 153 aa, Homo sapiens [sequence omitted]

SEQ ID NO: 2 - DNA, 614 bp, Homo sapiens
gatcgttagcttctcctgataaactaattgcctcacattgtcactgcaaatcgacacctattaatgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctgaatattttaatttatgagtttttgatagctttattttttaagtatttatatatttataactcatcataaaataaagtatatatagaatct

SEQ ID NO: 3 - DNA, 597 bp, Homo sapiens
ttctcctgtccggatgcgcagggcagggctgaccgtcgagctgcacccacagcaggctgcctttggtgactcaccgggtgaacgggggcattgcgaggcatcccctccctgggtttggctcctgcccacgggcctgacagtagaaatcacaggctgtgagacagctggagcccagctctgcttgaacctattttaggtctctgatccccgcttcctctttagactcccctagagctcagccagtgctcaacctgaggctgggggtctctgaggaagagtgagttggagctgaggggtctggggctgtcccctgagagaggggccagaggcagtgtcaagagccgggcagtctgattgtggctcaccctccatcactcccaggggcccctggcccagcagccgcagctcccaaccacatatcctctggggtttggcctacggagctggggcggatgacccccaaatagccctggcagattccccctagacccgcccgcaccatggtcaggcatgcccctcctcatcgctgggcacagcccagagggtataaacagtgctggaggctggcggggcaggccagctgagtcctgagcagca

SEQ ID NO: 4 - DNA, 2700 bp, Mus musculus
tctagaaagcactgttcctttaaaatcattcaccacctctggctcctacaatcttcctgtcctcccttccacacagatccctgagccttgaggagagggctgtgataaatcatcccctttggagtgagcagtctgaagtctctcattctccatgcactgtcttattccgtcccgcgggattcagttattcgtgggtgcgagggggaccacgaacctggaaggaaatgggaggaaaagaaagagagcggacgaccaagtagattgaacatatcaaggtctcgtttattaggctgaggtgccttctttttaaagcatacatcacggggaatatgggaggggtcgagggagaattatacaaagaacaaagaagtgggcatctgctgacatgggggccgaagtcaggcgccaggcagcgggcactctggattttatctctggaacattgatcctccttgacagccttgggggtcaggctgggctcaggcgtaactcatgtccttggatggcacgggaactcaggaagagatagggaagaggggactataattcagcttttacagcctcaggtgccaagaaaggaatagggaggaaggggggtgataaccagctcttagtacaaggccatttggcctgttagggagattgtgaagggctcactttctcacgggatggtctctgacactgtctggctgtgtgtctccccatctactgcaagactgggcttttctgatgaagtgtaagcctagtgagggtgccctgttcattagaagtcattttgcagtcactcagcagaatattagtagtgggtttctttccccctgagagctcacaacctgtctagtctcgggttcttagcaccgtgaataattctattttcagaagttaacatccttcccctcagacacctttgaagcttgtgggtgtttgggtttctgtgccctctacctgcacgtctctccatacccaactgtgagcatttgaaagcgtgtgctagagtttcttgtttagctccccatgtcctataaaacactttggtttggtagagaactgagcagttcaaactttgctcaactgagcttatgggggtgaattgaatacaagcaaataaaaggagcttattcaacttctcttttgtggttctctattttatttttaaatgctgaaatacttttctttagctaaatcatctgaagaatctaacagagtcactactctggcaacaatactggacaacaatggcatttattgatttctgtaaagtagaagtcaacagagaagaatatggggataaagaatatagggataaagaagacaaccaaccagagctcccagggtctaaaccaccaaccagggagtacacatggagggacccatggctccatctgtatatgtagcagaggatggcctagtccatcatcaatgggatgagaggcccttggtcccatgaaggcctgatatcccagtgtcggggaatttgagggcagggaggagagagtggatgggtaggtgggggaacaccctcatagaagcaggagggggggtgggatagggggttttggggtgtgggaattgggaaaggggataaaccttgaaacgtaaataaataaaatatccaataaaaaaatcttctggaaaagaaaagatatacaaaatacaaaggcagtttcctttgcaaacttaggaaatgttcagtttgccaatgcatgcagtaagtttattttccagtaattattcaataaccatgaactgctctctggcagtgctagtaattattctctactcataggaaaaaaattacataagaagacgactagaaataagattatacgatgtgcagtggcctcatttacacagcaaagggccacataggggataatcccaaggacttgttctatgaaaggttacatcagctccttggtctcaacctcgaacgctgtaacgttcacagtcagcattgtgctttagcaaagcttaggtaatctgactggtttaataatatcagttttgacttacaagcctctgaaatatgtttcagggagaaatataaaggaatcaatattaaactatctcttggcatcaactcatttcctaattcagtacttttagacccatgcagtgctgtgtgaaagccagctttcctttctttcaacacagtgaaaacctgtatcattgtgaaagcttaaatgcttaagtcttttgctatttattttatttgaaatgcagtatattattatatatattcagaactctaactaccatcttctcctcacccttcaattaaatcccacaatgcaagcctcttggcagaaggcccacctttcatgtttattcaactgaggctgaatcttgaaaatgtgttgaagtttgggattctctggtgagaacccacagcctgacgttgtgctggccacagctgtgattggctgttgagaggcggagaagggtttatagtcagcaagagcaagtgaatgagtgagtgacagccgggagaacaatccgtgccactcactcgactcgagccaaggacctggccgaaaggaaggttaaggtaatgggcaaggacctcacagccaggtaatgggcaaggacctcacagccaggcacctcagtcttccctgtgtggctttggcttggagtttgtagctgcagcatggatcttactgcacagtgcacagtggctctagttgaacttttgcttgc

SEQ ID NO: 5 - DNA, 1093 bp, Rattus norvegicus
aagcttagggaacattcagcctgccaacatacgcgggaagtttattttccagtgatcctttcaatggccgtggaactgctttctggcagtgctagtaattcttctctcctcagagggaaagatacataggaagaggacttagaaataagcctgagagtatacagcgcttgatgacctcactcgcacaacgaaaggccatgtcccggatgatgccaactactttgttcgatgagagttaaatcagcttcttggtctgagcctcaaatgttgtagctttcacagtcagcacagttagcaaagccttggcagcccggctggctttacaatactgattctgacttacgagcctctgaaatgcatttcagaaaggaatataaagggatcttcactgaacacctcttgtcatcaactcgtttcctaattcagtgcttttaggctcgggcagtgctgtgtttaacagaggctagttttcctttctttcaacatagtaaaaacctgtatcattgtgaaagtttaaatgcttaagtcgtttgccatttagtttatttgaaatgcagtgtattattatagatattcagaactctaactaccatcttctcctcagccttcaattaaatcccacaatgcgacctcttggcagcaggcgcgcctttcatgtttattcaactgaggctgagtcttgaaaacgtgttgtagttacggattttctggtgagaacccacagcctgacgtcgcaccggccgtgaccgtgattggctgctgagaggagaagaagggtttataggtcagcaagagcgagtgaatgggtgagaggcagccgggagaacaatccgtgccactcactcacttgctctctccagccaggactgccgaaggtaaggtaatgggccagcacctcacagccacctgcctcaggcttcctgtgtggctttggcttggaatttgtcgttgaagcatggatcttactgcttggtgcacaatggctctggttgaactttagcttgctgtgaaatgggacctctgagtttaggttctttccaaagaccaggctgggtaacgtaagcatgcagttaaactgcttcagattggtacc

SEQ ID NO: 6 - DNA, 1627 bp, Mus musculus
gaattcttttcccattggtaacgtaaaagaccactacttaattgagttagcttaggctcaacaaacagactttatacaacttaacttccttcacatttatgaaaaattaatcagtatcggcactgagaaggcagaaacaggtagaactccatgagtttcaggccagcctgatctacataggaattctaggacaagcagggctaggtagagataccctatctcaaaaaaccaaaacccaaaaacattacgtttaagcagatttagttttgaccctaaatgtttgtcttagtgaaggtcccaaatgctcttagcaaatgtttctttgtgtagttggagagtgttgtgtgctaatacagctatcaagcacttctgtttagacaccgaagatcttcttaactctccatcaggtctggagagctgttcaaatctgctattacaaccaagttaggaagaggaaggcaattcctgaggaaagtggcattcttaaatatgattggccctttaagatgctcaaagaaccaagaaccatgcagtgtaaataatagcaaagtgtttactatggaagtgcagcttcgaggaaactcccttcctatcactggaacctgtccaatccctacctacatgaatatgttgtttaattctctcagtataaagctctgaagatgctgttgctggatagtgatttaatatttctgatcatatgtgtttgacatctttcagtagtgtgacataaaaacatggacacatccctaagctggtacacagagactccaattgcctagtgtggagctcataagctagagaaatggctcagggatcatcttgtatatccagggctcgagagaatgatgggttcaggcaagtactttttcctttctggaagcacagcctgttttcctattctgtactctatagtttacacatatagtggagcaaagaatgaaagctgtgtctgtggtgtgtgtgtgtgtgcactctgtacttacgcatagataccttacaccatgtttcacctttggaacagctatttttaaatttagtttgtattaaattaatagattataaagaaaaacccaaaacctttatgtcagtgtttagattaaatcagaaaggtttcctgaagttactgtttataaattcttttaaagatcccttaggcagtgtcaagactgttgcatgcggacagccgcttgaattatagcgcaccaactttaatatgtacctcaggaatgataggggtcttaaatagccagtcgtatttactagagaaacctagagttttcttagattgccgacctaagcaagaggagaaatgcagggtgacagagtctaagtggctcttttcagatatatcacactgattatctatatttaagacacaaaacagtcttccaggagctatttaattaagtgaaagtaagtctagtccttttggaaccaaaggtctcagtgagccaacgtaccggcgagcgagggagtggggcgttattacagcctcataggcacactgactctttaaacccccacatcagggatcctaagcagtgattggttgagaaaattatcaaactgaatttaaatttcagcaggtacaaaattgtcacgcaaaaagcccaggacagtgtgc

SEQ ID NO: 7 - DNA, 3240 bp, Mus musculus
gtaagatggactccctcctgccaggagccaactgtctcctgttgagagaatctccagctgcagagatgagggtgacttgggataaagtttttaactcttcaggtctacactatatattaaagataatgtgtgattcaggaaggggtgctaagccatctgatgagaccatctgataagacgacgaatcactggggagcagaactgattttgccccagtatattgttgagactttatctcctataggaaaaacctaagatgaaacaaacattctaattgtattaattaaaaaaaaacagtacctgaagggttttatgtatagttctctatagctctatttttgttattttcattcaggaaaatacttttaagagctataaacctagtcaaaggtgttttacagccttgtccttggaatgttgggagtgttgggatttaacaaatgagaatcacacactgtcttcctcttcgagacagagacatggatgatgcagtgtccaaacaccagctcttcctgaaaaataagctgggtttgggggtttgatttaatcatggctcttcatgatttcaaggtctgcctagtgtttatgattaaagctctatggcgaaaagaattgtggttcctcccagggctcagtatctgcctgatattaacttccgatgttcactgactggacctaataaataaatctccatttaaacttagtatcttgactcagagtcaacttaggatctgggagcgtaattttctggcatgtgatgtgaagtttctaaaagtagacgctcaaacagttttatgtagaaaacacacagatctgtcaagctgatttttcagctccaaatttcatgataataggtttagggaaaacaaagacatattgcctcaagttggcaaaaattgaggtggaaatttgaatgtggtcactttgaatggttttgatttaagaaaaaatagataacttgtattgtaaatatctttaaaatatttttattcattccctgagaaatttgtgtggtatgttctgattgctctccccagatctgcctttgttctttactcacacaactttgtgctctttttgtaaagaaacaaaacaagagccatgcacaccagtttgtgctcctcaaatgtactcagctgtgtggccatctgctgggttctggttgccttaccaggggctacattcttggagaacactgcctttccttttttcccaccacctattgttaattgttcttcatgtccagctttcctctccttgctgggatttggtctgacttgggcttgcacggtcgggtgcaggctgtcagaagcgctgtgaagatagctcgggtagtttaagtctacctcaggcattccaacaaggccctcacaatgaggctttgcgtttcctggtcttcttagtgagtgatatattcattctaactggctattcatacatttcatctagtgtggggcaataaatgggacaatttaaaggagcctcaattctaatgactggttatttccaccagggtctttgatatggttgacctgccttgccaacaggtgcaagtatcatatatgtcagtgctggagtggaaatgtggtgtgtgtgtgtgtgtgtgtccgtgtgtgtgtgtgtgtgtgtgtgtgtgtaaggagggatggaaggtggatggtgggagacaggaattctcagatggtcagatttcagtttagaaattatatgtgtgtgtgtgtgtgtctgtctgtctgtctggactttattgcaggtacctttccaggaccagggatccccagttcacactcggtttagagttgccaagctcaagtataagcttggcttggtagacagatggccttcacctcaactcctggccctggggctttgtctcaaggcacctcattttagtttgtagaataattgaagggaccccagcttttcttagctttctcttgacagctataaggaagggtgaagcatctttttcagagatcctagaattgtgttctcacttctgtcaagtaataaacaatatatattcattgatgttttattctattcccctattaaccttggattttaatcaaggacattttatgatgtgcaaggtggtaatcattaattcttgtggaaggtcacaagataggagaaaacaattctttctatagtaaaacaccatgatacaaataaatttagttttagaaaatgggaacctgaagttttgattcacatagatttttatagttttacaggctccattccaatgtatgaaaaatatgtatctgattctgtgaatttgcattgcaaagggtgaaagatttcactcttgaagcctctctccttcagctcctccctcagtccgagactgcatagtgcccgggtaagggtggggtgtcctttgtcctcaggagtgcttgttcagcagcaggctctgcaaggtgacctttgctttgctcagaagacactgatgatcaagatgctggcgtgggctccgagacctgatgccagtgaggaggaagatggggtagctaggcaacttcaaaacagtgcaatgtgctgccagcatcgagcgagcggagggtgcacaagctgatgctgtgtgaggaagggagctaaagatgccttcagaaagctttttgggggtgattcttctgccaacccctaggatattgtgagctacagagttattaaaccagactgaggaaacaaaagcccaataaagctattgaaagtgcccaagctcagagagcagatagcaggggaaggatttgaattcagggatctgaaaccaaatcctgtgttctctctcctagcctaaactctctcttccttaaacactgtaagaggaagatttcttcctcttactgggataacgcccaattctatatagaccaggtgggaaattacaagtgctttatcatttacaatctacttttagttaatgatgcttaaagctagcccaggagagacgttaccctcatggataacagcatagggccagagccacgagctatgtactctgtatcttcatggctgttgcttccacaggcaggtagagtcagaagccatgacagtcctgagcatgcagaggcccccacatacccaggtttatttctggaacctggggtgttttctcacattagtactttctccttgtcctagaaaagggccaaatgtaagaccaaaatattggggtactgtggctgtcatctttcatcttatgacccgttttgtggtgttctttgttctaaacag